(19) United States
(12) Patent Application Publication
(10) Pub. No.: US 2007/0019778 A1
(43) Pub. Date: Jan. 25, 2007

(54) Voxel histogram analysis for measurement of plaque

(76) Inventors: Melvin E. Clouse, Brookline, MA (US); Vassilios Raptopoulos, Brookline, MA (US); Shezhang Lin, Shrewsbury, MA (US); Adeel Sabir, Boston, MA (US)

Correspondence Address: Faustino A. Lichauco;FISH & RICHARDSON P.C., 225 Franklin Street, Boston, MA, 02110, US

(21) Appl. No.: 11/187,624

(22) Filed: Jul. 22, 2005

(51) Int. Cl. H05G 1/60; A61B 6/00; G01N 23/00; G21K 1/12

(52) U.S. Cl. 378/4

(57) ABSTRACT

A method for estimating a volume of plaque in a blood vessel includes identifying a range of voxel densities and imaging a selected volume of the blood vessel. Within the selected volume, the number of voxels having a density within the range is estimated. At least in part on the basis of the estimated number of voxels, the volume of plaque is estimated.

### FIELD OF INVENTION

The invention relates to radiology, and in particular, to measurement of plaque volume.

### BACKGROUND

Non-calcified plaque that forms on arterial walls may migrate and interfere with circulation. This poses grave risks for the patient. It is therefore desirable to identify the presence of such plaque, to determine its volume, and to observe its spatial distribution.

Known methods of determining the spatial distribution and volume of non-calcified plaque are either invasive, inaccurate, or both. For example, one known method is to insert an ultrasound probe into the blood vessels and to obtain ultrasound images. These ultrasound images are difficult for even an experienced radiologist to interpret reliably.

### SUMMARY

The invention is based on the recognition that three-dimensional imaging techniques are adaptable to measurement of non-calcified plaque volume and distribution.

In one aspect, the invention features a method for estimating a volume of plaque in a blood vessel by selecting a first range of voxel densities; imaging a selected volume of the blood vessel; at least in part on the basis of the resulting image, estimating the number of voxels having a density within the first range; and at least in part on the basis of that estimate, estimating the volume of plaque.

Some embodiments also include storing data indicative of locations of the voxels; and, at least in part on the basis of that stored data, displaying an image showing locations of plaque in the blood vessel.

Other embodiments also include selecting a second range of voxel densities; and suppressing display of voxels whose densities are within the second range. Among these embodiments are those in which selecting the second range includes selecting the range to be consistent with the density of arterial wall.

In some embodiments, estimating the number of voxels includes dividing the volume into a plurality of slices; and for each slice, estimating a number of voxels having a density within the first range. Among these embodiments are those in which estimating a number of voxels having a density within the first range includes defining a path through the slice, the path intersecting a set of voxels, and counting the number of voxels in the set of voxels that have a density within the first range.

In some embodiments, selecting a first range includes selecting the range to be consistent with the density of non-calcified plaque.

Other embodiments include those in which imaging a selected volume includes exposing the volume to x-rays; generating data representative of x-rays that have interacted with matter within the volume; and at least in part on the basis of the data, constructing an image of matter within the volume.

In another aspect, the invention features a method for estimating a volume of a feature in a body lumen by selecting a first range of voxel densities; imaging a selected volume containing the lumen; at least in part on the basis of the resulting image, estimating the number of voxels having a density within the first range; and at least in part on the basis of the resulting estimate, estimating the volume of feature.

Embodiments include those in which the feature is selected to be plaque, and those in which the feature is selected to be arterial wall.

In yet other embodiments, imaging the selected volume includes multiple-detector computer tomographic imaging of the selected volume.

Additional embodiments include those in which selecting a first range includes selecting the range to be consistent with the density of non-calcified plaque. Among these are embodiments in which selecting the second range includes selecting the range to be consistent with the density of arterial wall.

In other embodiments, imaging a selected volume includes exposing the volume to x-rays; generating data representative of x-rays that have interacted with matter within the volume; and at least in part on the basis of the data, constructing an image of matter within the volume.

Yet another aspect of the invention features a computer-readable medium having encoded thereon software for executing any of the foregoing methods.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

These and other features of the invention will be apparent from the following detailed description and the accompanying figures, in which:

### BRIEF DESCRIPTION OF THE FIGURES

FIG. 1 shows a system for determining the volume of non-calcified plaque;

FIG. 2 is a functional block diagram of software executing on the data processing system of FIG. 1;

FIG. 3 is a density profile across a normal blood vessel;

FIG. 4 is a density profile across a blood vessel afflicted by non-calcified plaque;

FIG. 5A is a three-dimensional image of a blood vessel.

FIG. 5B is an image of the blood vessel shown in FIG. 5A, but with display of the wall suppressed.

FIGS. 6A-6D are images of the blood vessel shown in FIG. 5B but with the display of various combinations of features suppressed.

### DETAILED DESCRIPTION

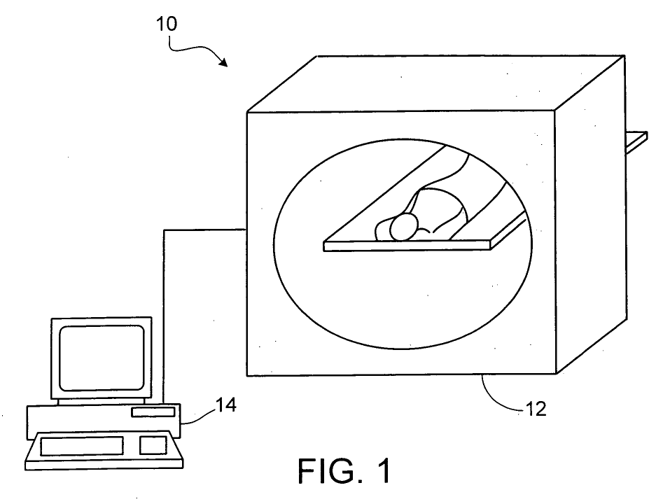

Referring to FIG. 1, a system 10 for determining the volume of non-calcified plaque includes an image acquisition unit 12 in communication with a data processing system 14. A suitable image acquisition unit 12 is a multiple-detector computerized tomography (“MDCT”) unit. Exemplary MDCT units are manufactured by Toshiba Medical Systems, Inc., Tustin, Calif. 92780, Siemens Medical Systems, Inc., General Electric, Inc., and Phillips Medical Systems, Inc.

The image acquisition unit 12 exposes the patient to radiation and detects the extent to which the patient's tissues interacts with that radiation. This results in image data that is ultimately provided to the data processing system 14.

For example, in the case of an MDCT unit, the patient is injected with a contrast agent, such as OPTIRAY 350, IOVERSOL, available from Mallinckrodt, Inc., of St. Louis, Mo., and a selected volume within the patient is exposed to X-rays. The extent of interaction with the X-rays results in a density value at each voxel within the selected volume of the patient.

The data processing system 14 includes a processor, a memory, and a mass storage element that cooperate to execute software for determining the volume of non-calcified plaque in the selected volume of the patient. The software does so on the basis of image data provided by the image acquisition unit. In most cases, the selected volume encompasses a segment of a blood vessel.

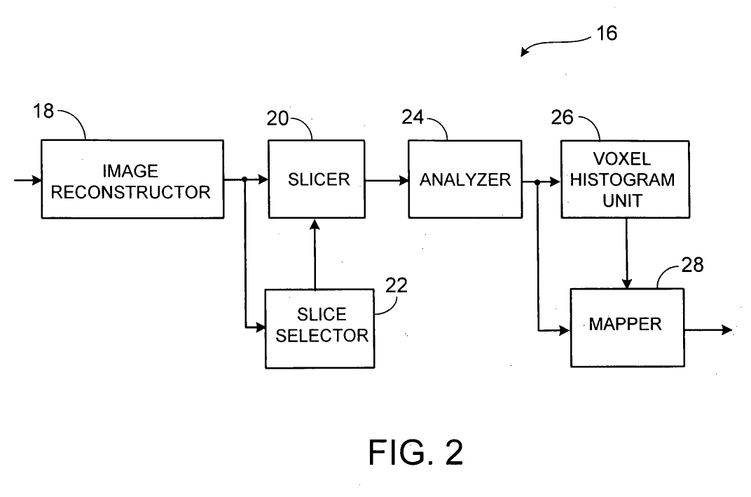

FIG. 2 shows a functional block diagram of software 16 that is adaptable for measurement of plaque volume. Suitable software includes Analyze version 6, which is developed and maintained by the Mayo Clinic, of Rochester, Minn.

The software includes an image reconstruction module 18 for using image data to generate data representing a three-dimensional image of the selected volume. This reconstructed image data is provided to a slicer 20.

The slicer 20 extracts slices from the reconstructed image data. These slices are two-dimensional surfaces that are offset from each other. To avoid mathematical complexity, the two dimensional surfaces are preferably planes that are parallel to each other.

A slice-selector 22 in communication with the slicer 20 uses the reconstructed image data to define the number of slices, and their locations. In some cases, the slice-selector 22 chooses slices that are equally spaced from each other. However, in other cases, for example where variable resolution is sought, the slice-selector 22 chooses slices that are close to each other within a region of interest and further from each other elsewhere. In other cases, the slice-selector 22 is adaptive, and determines how close the slices are to each other on the basis of whether plaque has been or is expected to be detected in a particular region.

Data representative of voxels intersecting a slice is then provided to an analyzer 24. The analyzer 24 defines a path that traverses a slice, or a portion thereof, and collects data representing the density of the voxels along that path. As used herein, the density of a voxel, or “voxel density,” refers to a characteristic of an image at that voxel. It is not intended to refer to the number of voxels in a particular area or volume. In the case of MDCT, that characteristic reflects the extent to which X-rays pass through the medium contained within the voxel.

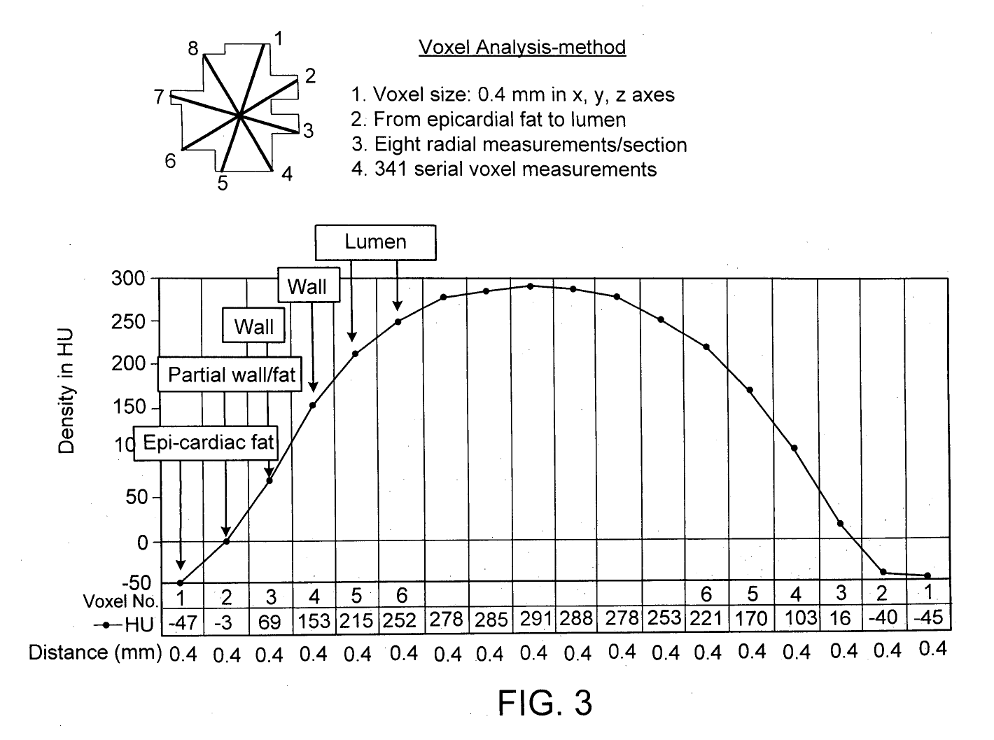

Since different media have different densities associated with them, it is often possible to identify the particular medium contained within a voxel on the basis of a density associated with that voxel. FIG. 3 shows a typical plot, referred to herein as a “density profile,” of voxel density as a function of position along a path. In the case shown in FIG. 3, the path traverses the diameter of a relatively healthy blood vessel.

In FIG. 3, there are 341 voxels along the path, with each voxel being a 400 micron cube. The particular blood vessel is a coronary artery. Starting at the left side of the figure, one notes voxels of relatively low density (typically −42±19 to 1±12 HU) characteristic of epicardiac fat. As one proceeds further toward the right, the density rises to levels consistent with those found for arterial walls (typically 67 to 144 HU). As one proceeds further rightward, the density rises to levels consistent with those found for contrast agents. The attainment of these levels marks the end of the arterial wall and the beginning of the lumen. As one proceeds further to the right in FIG. 3, the voxel densities begin to decrease in the reverse order.

The path traversed in FIG. 3 is but one of many possible paths traversing the diameter of the blood vessel. To provide a better picture of a blood vessel, the analyzer 24 repeats the foregoing process for several different paths. The precise number of paths across the blood vessel depends on the desired resolution and on processing constraints. In most cases, each path is defined by a pair of a radial lines that extend outward from the centre of the lumen and through the wall of the lumen.

The radial lines are circumferentially offset from each other. By default, the angles between radial lines are equal. However, like the number of slices, the number of radial lines and the respective circumferential angles that define their directions can be changed to suit the circumstances.

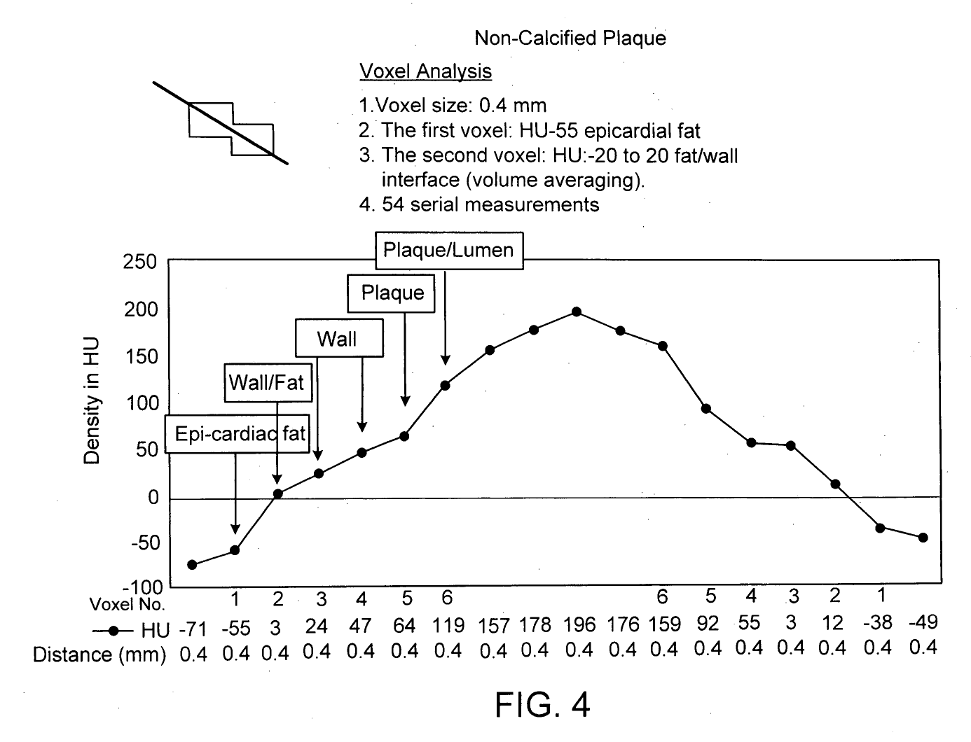

FIG. 4 shows the output of an analyzer 24 that has generated a voxel density profile for a path across a coronary artery of a different patient. In this case, the artery is one afficted with non-calcified plaque deposits. The density profile begins as it did in FIG. 3, with voxels whose densities are characteristic of epicardiac fat and the arterial wall. However, traversal of the wall does not result in a rise in density, as was the case in FIG. 3. Instead, the density rises to levels consistent with the presence of non-calcified plaque. Only when one has proceeded far enough into the lumen does the density rise to levels associated with the contrast medium.

Referring back to FIG. 2, voxel density data is provided to a voxel histogram unit 26 that sorts the voxels into particular density bins, with each density bin being defined by an upper and lower threshold. The voxel histogram unit 26 thus accumulates data indicative of how much non-calcified plaque is present in a particular slice and provides that data to a mapper 28.

The foregoing procedure of determining voxel density profiles across multiple paths in a particular slice results in data for only a two-dimensional surface. To provide three-dimensional data, the procedure is repeated for different slices. This additional data across different slices enables the mapper 28 to generate a three-dimensional image of a selected portion of a blood vessel, and to calculate the total volume of non-calcified plaque within that selected portion.

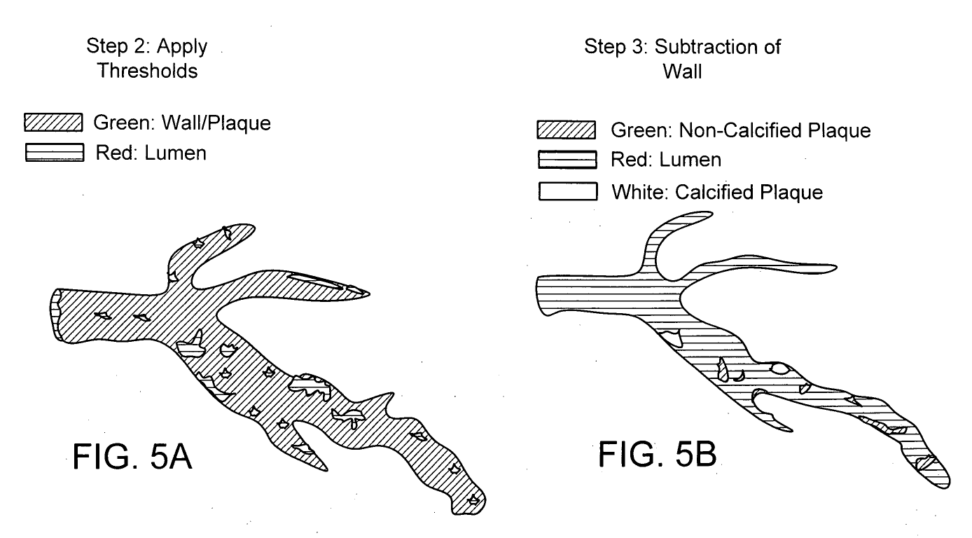

FIG. 5A shows a three-dimensional image of a blood vessel as provided by the mapper 28. In FIG. 5A, all voxels are displayed. As a result, the wall obscures any structures within the blood vessel. Since voxels associated with a wall are characterized by particular densities, and since voxels have been sorted into bins representing various density ranges, the mapper 28 can readily suppress display of all voxels sorted into bins whose densities are consistent with that of the wall. This suppression, or filtering, results in the image of FIG. 5B, in which one can effectively see through the wall and into the lumen.

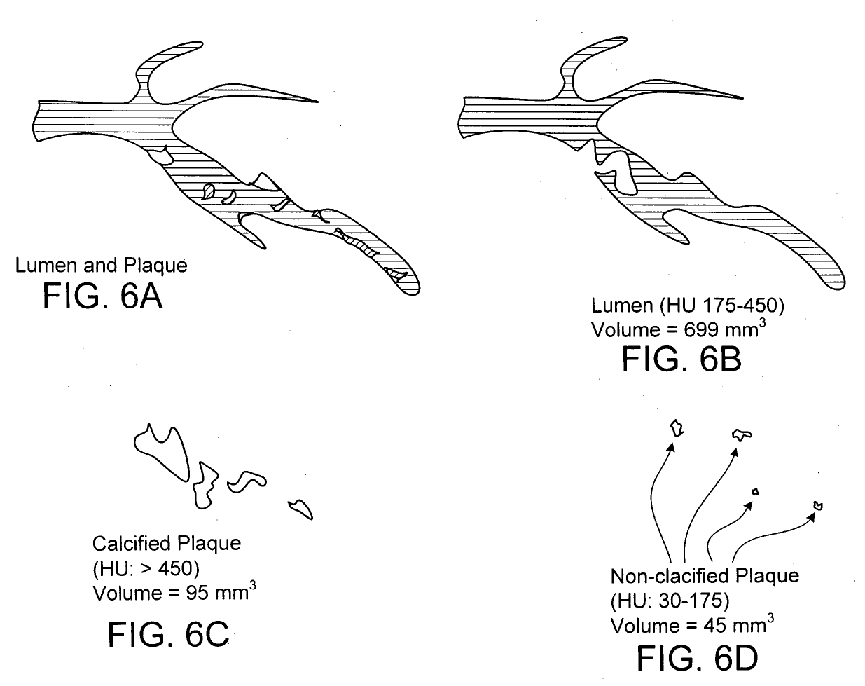

That the voxels have been sorted into bins by density values results in additional flexibility. For example, in FIG. 6A, the mapper 28 displays both the lumen and the plaque; in FIG. 6B, the mapper 28 suppresses display of all voxels except those having a density range consistent with being in the lumen. In FIG. 6C the mapper 28 suppresses display of all voxels except those having a density range consistent with being calcified plaque; and in FIG. 6D, the mapper 28 suppresses display of all voxels except those having a density range consistent with being non-calcified plaque.

### EXAMPLES

### Example 1

From eleven coronary CTAs, cross-sectional images of 55 blood vessels were obtained. Of these, 48 were images of normal vessels and 7 were images of vessels having non-calcified plaque in 7 proximal arterial segments (RCA-2 segments, LM-1, LAD-2, LCX-2).

Eight radii were defined for the normal vessels, with each radius being circumferentially offset from its neighboring radii by 45 degrees. This configuration of radii was used in all examples disclosed herein.

According to the histogram of each line, densities of 6 consecutive isotropic voxels (0.4 mm on each side) were recorded. The voxel whose density was nearest to 0 was defined as the second voxel in each series.

The normal wall thickness proved to be 2 voxels (0.8 mm). The density at the interface between the epicardiac fat and the wall was 30 HU (Hounsfield Units). The density at the interface between the wall and the lumen was 175 HU. These measured parameters were used for the three-dimensional processing of 22 coronary segments with non-calcified or mixed plaque and for three-dimensional processing of 16 apparently normal vessels. Data for the normal vessels was used to abstract the vessel and to subtract the wall, lumen and calcified plaque. The remaining voxels, which had densities between 30 and 174 HU, were used to calculate the non-calcified plaque volume.

Densities (mean ± standard deviation in HU) of six voxels across a normal wall were measured to be −42±19 (epi-cardiac fat), −2±19 (partial fat/wall), 67±38 (wall), 144±57 (wall), 211±65 (lumen), and 255±63 (lumen, p<0.01 ANOVA). The correlated voxel densities across the wall/plaque interface were: −34±16 (fat), 1±12 (fat/wall), 46±27 (wall), 92±53 (wall), 133±77 (plaque), and 162±88 (plaque). This data indicated an increased thickness related to plaque. The wall thicknesses along the eight radii defined in normal vessels were not significantly different (p>0.05 ANOVA). Sensitivity in quantifying non-calcified plaque volume (39±12 mm³) was 100% (22/22), and specificity was 87.5% (14/16).

### Example 2

Voxel analysis of coronary wall density and thickness was performed on 48 cross-sectional images of normal vessels in six proximal coronary segments (RCA-2 segments, LM-1, LAD-2, LCX-1) imaged by 16/64-MDCTA. Voxel histograms were obtained along eight radii extending between outside the wall and the lumen center. HU density measurements of six consecutive isotropic 0.4 mm cubic voxels were recorded. The second voxel, the density of which was nearest to 0, was used to defined the outer wall.

Data analysis revealed the existence of two connected voxels whose densities were significantly different from those of epicardiac fat and lumen. The densities at the interfaces between the epicardiac fat and the wall and between the wall and the lumen were measured to be 30 HU and 175 HU, respectively. With these parameters, two processing methods were used to measure the intra-luminal volume of each of fifteen selected normal arterial segments. In the “subtraction method,” lumen volume was determined by subtracting the two voxels that represented the wall, and counting the remaining voxels as representing lumen. In the “threshold method,” lumen volume was determined by disregarding all voxels having a density below a threshold, in this case 175 HU, and counting only those voxels having densities in excess of the threshold. The resulting count would then represent lumen volume.

Densities (mean±standard deviation in HU) of six voxels across normal wall were: −42±19 (epicardiac fat), −2±19 (partial fat/wall), 67±38 (wall), 144±57 (wall), 211±65 (lumen), and 255±63 (lumen, p<0.01 ANOVA). The wall thicknesses along the eight radii were essentially the same (p>0.05 ANOVA). The intra-luminal volumes measured by the subtraction method and by the threshold method were 220±116 and 223±109 mm³, respectively (p>0.05 paired t-test), and were statistically correlated (r=0.96).

### Example 3

Forty subjects (mean age 59.9 years, 76% males) underwent contrast enhanced MDCTA. Advanced reconstructions were performed, using Vitrea2, ADW4.2, Analyze image reconstruction software, on 11 of the subjects to obtain 48 normal cross-sectional images and 7 images showing non-calcified plaque in coronary segments RCA 1, 2, LM 5, LAD 6, 7, and LCX 11, 12 as demonstrated by CCA and MDCTA.

Attenuation values of 6 consecutive 0.4 mm isotropic voxels (identified as voxels A-F) were measured along eight radii as described in example 1. Voxel A lay outside the wall, in epicardiac fat; voxel B lay at the interface between the epicardiac fat and the wall; voxels C and D were within the wall; and voxels E and F lay within the lumen. A total of 395 serial measurements of each voxel were performed to obtain their HU densities. Of these, 341 were performed in normal sections, and 54 were performed in plaque-containing sections.

HU values (mean±standard deviation of each voxel) were as follows:

| | Normal | Plaque |
|---|---|---|
| Voxel A | −42 ± 19 | −34 ± 16 |
| Voxel B | −2 ± 19 | 1 ± 12 |
| Voxel C | 67 ± 38 | 46 ± 27 |
| Voxel D | 144 ± 47 | 95 ± 53 |
| Voxel E | 211 ± 65 | 133 ± 77 |
| Voxel E | 255 ± 63 | 162 ± 88 |

The greatest increases in mean HU value were between voxels B and C (p<0.05) and between voxels D and E (p<0.05). Since these represented the outer and inner wall boundaries, their averages were calculated to compensate for partial volume effects. This resulted in mean attenuation values of 30 HU and 175 HU respectively. Using these values the average wall thickness for normal vessels was estimated to be 2±1 voxels, which corresponds to 0.8±0.4 mm.

These 48 normal and 8 abnormal vessels were analyzed using the subtraction method discussed in connection with example 2. Eighteen non-calcified plaques in the RCA, LM and LAD, coronary segments were analyzed. These plaques had volumes ranging from 7 mm³ to 139 mm³. Four of these plaques had densities in the lipid range; the remaining fourteen had densities in the fibrotic range.

### Example 4

Out of forty subjects who underwent contrast enhanced coronary CTA with 16 and 64-slice scanners, eleven subjects, nine of whom were male, had non-calcified plaque. These subjects underwent advanced reconstructions on six coronary segments (AHA segments #1, 2, 5, 6, 7 & 11) to obtain 48 normal cross-sectional images and 8 cross-sections with non-calcified plaque. The resulting images were evaluated with voxel histograms to determine the attenuation values of wall, non-calcified plaque, and lumen. A minimum of seven consecutive, isotropic, 0.4 mm cubic voxels, designated voxels A-G, were measured along each of eight radii as described in connection with example 1. The voxel having an attenuation value closest to 0 was voxel C. Voxels A and B lay outside the wall, in epicardiac fat; voxels C, D, and E spanned the wall and/or plaque; and voxels F and G lay within the lumen or plaque, depending on the particular cross-section.

A total of 2,388 voxels in normal sections and 375 voxels in plaque-containing sections were measured. Density values (mean±standard deviation), measured in HU, for the voxels were as follows:

| | Normal | Plaque |
|---|---|---|
| Voxel A | −58 ± 27 | −51 ± 18 |
| Voxel B | −42 ± 19 | −34 ± 16 |
| Voxel C | −2 ± 19 | 1 ± 12 |
| Voxel D | 66 ± 38 | 45 ± 27 |
| Voxel E | 144 ± 57 | 92 ± 53 |
| Voxel F | 210 ± 65 | 133 ± 77 |
| Voxel G | 255 ± 63 | 162 ± 88 |

In normal sections, the mean attenuation values of the voxels were significantly different from each other. The greatest increase in mean HU value was observed at the interfaces between voxels C and D, and between voxels E and F (p<0.05). Since these two interfaces represented the outer and inner walls, their averages were corrected to compensate for any partial volume effects. This resulted in mean attenuation values of 30 HU and 175 HU, respectively. The average thickness of a normal wall was 2±1 voxels (0.8±0.4 mm). For comparison, the literature discloses a normal wall thickness of 0.88±0.2 mm, as measured using HFEE (“High Frequency Epicardial Echocardiography”) and MRI (“Magnetic Resonance Imaging”).

It is evident that those skilled in the art may now make numerous modifications of and departures from the apparatus and techniques herein disclosed without departing from the inventive concepts. Consequently, the invention is to be construed as embracing each every novel feature and novel combination of features present in or possessed by the apparatus and techniques herein disclosed and limited only by the spirit and scope of the appended claims.

We claim:

1. A method for estimating a volume of plaque in a blood vessel, the method comprising:
selecting a first range of voxel densities;
imaging a selected volume of the blood vessel;
at least in part on the basis of the resulting image, estimating the number of voxels having a density within the first range; and
at least in part on the basis of the estimate of the number of voxels, estimating the volume of plaque.

2. The method of claim 1, further comprising
storing data indicative of locations of the voxels; and
at least in part on the basis of the stored data, displaying an image showing locations of plaque in the blood vessel.

3. The method of claim 2, further comprising:
selecting a second range of voxel densities; and
suppressing display of voxels whose densities are within the second range.

4. The method of claim 3, wherein selecting the second range comprises selecting the range to be consistent with the density of arterial wall.

5. The method of claim 1, wherein estimating the number of voxels comprises
dividing the volume into a plurality of slices; and
for each slice, estimating a number of voxels having a density within the first range.

6. The method of claim 5, wherein estimating a number of voxels having a density within the first range comprises:
defining a path through the slice, the path intersecting a set of voxels,
counting the number of voxels in the set of voxels that have a density within the first range.

7. The method of claim 1, wherein selecting a first range comprises selecting the range to be consistent with the density of non-calcified plaque.

8. The method of claim 1, wherein imaging a selected volume comprises
exposing the volume to x-rays;
generating data representative of x-rays that have interacted with matter within the volume; and
on the basis of the data, constructing an image of matter within the volume.

9. A computer-readable medium having encoded thereon software for executing the method of claim 1.

10. A method for estimating a volume of a feature in a body lumen, the method comprising:
selecting a first range of voxel densities;
imaging a selected volume containing the lumen;
at least in part on the basis of the resulting image, estimating the number of voxels having a density within the first range; and
at least in part on the basis of the estimate, estimating the volume of feature.

11. The method of claim 10, further comprising selecting the feature to be plaque.

12. The method of claim 10, further comprising selecting the feature to be arterial wall.

13. The method of claim 10, wherein imaging the selected volume comprises multiple-detector computer tomographic imaging of the selected volume.

14. The method of claim 10, wherein selecting a first range comprises selecting the range to be consistent with the density of non-calcified plaque.

15. The method of claim 14, wherein selecting the second range comprises selecting the range to be consistent with the density of arterial wall.

16. The method of claim 10, wherein imaging a selected volume comprises
exposing the volume to x-rays;
generating data representative of x-rays that have interacted with matter within the volume; and
at least in part on the basis of the data, constructing an image of matter within the volume.

17. A computer-readable medium having encoded thereon software for executing the method of claim 10.
